(19) United States
(12) Patent Application Publication
(10) Pub. No.: US 2004/0186415 A1
(43) Pub. Date: Sep. 23, 2004

(54) Blood circuit with leak-safe features

(76) Inventors: Jeffrey H. Burbank, Boxford, MA (US); James Brugger, Newburyport, MA (US)

Correspondence Address: Proskauer Rose LLP, Room 17-25, 1585 Broadway, New York, NY, 10036, US

(21) Appl. No.: 10/393,209

(22) Filed: Mar. 20, 2003

(51) Int. Cl. A61M 37/00

(52) U.S. Cl. 604/6.16; 604/5.01

(57) ABSTRACT

A leak safe access needle and blood circuit are combined in a fluid circuit with the access needle configured as a double lumen access needle whose venous line is permanently attached to the fluid circuit. The arterial line has an air detection device. In this configuration, the only way the venous line can be disconnected is for the access needle to be withdrawn from the patient, which will necessarily result in disconnection of the arterial line, which in turn will be detected by air infiltration into the arterial line. This combination ensures that venous disconnects are revealed by the air detection. Other embodiments allow non-permanent connection of the venous line.

### FIELD OF THE INVENTION

The present invention relates to the detection of leaks, particularly disconnections in the venous line of a blood circuit.

### BACKGROUND

Many medical procedures involve the extraction and replacement of flowing blood from, and back into, a donor or patient. The reasons for doing this vary, but generally, they involve subjecting the blood to some process that cannot be carried out inside the body. When the blood is outside the patient it is conducted through machinery that processes the blood. The various processes include, but are not limited to, hemodialysis, hemofiltration, hemodiafiltration, blood and blood component collection, plasmaphresis, aphresis, and blood oxygenation.

One technique for extracorporeal blood processing employs a single “access,” for example a single needle in the vein of the patient or a fistula. A volume of blood is cyclically drawn through the access at one time, processed, and then returned through the same access at another time. Single access systems are uncommon because they limit the rate of processing to half the capacity permitted by the access. As a result, two-access systems, in which blood is drawn from a first access, called an arterial access, and returned through a second access, called a venous access, are much faster and more common. These accesses include catheters, catheters with subcutaneous ports, fistulas, and grafts.

The processes listed above, and others, often involve the movement of large amounts of blood at a very high rate. For example, 500 ml. of blood may be drawn out and replaced every minute, which is about 5% of the patient's entire supply. If a leak occurs in such a system, the patient could be drained of enough blood in a few minutes to cause loss of consciousness with death following soon thereafter. As a result, such extracorporeal blood circuits are normally used in very safe environments, such as hospitals and treatment centers, and attended by highly trained technicians and doctors nearby. Even with close supervision, a number of deaths occur in the United States every year due to undue blood loss from leaks.

Leaks present a very real risk. Leaks can occur for various reasons, among them: extraction of a needle, disconnection of a luer, poor manufacture of components, cuts in tubing, and leaks in a catheter. However, in terms of current technology, the most reliable solution to this risk, that of direct and constant trained supervision in a safe environment, has an enormous negative impact on the lifestyles of patients who require frequent treatment and on labor requirements of the institutions performing such therapies. Thus, there is a perennial need in the art for ultra-safe systems that can be used in a non-clinical setting and/or without the need for highly trained and expensive staff. Currently, there is great interest in ways of providing systems for patients to use at home. One of the risks for such systems is the danger of leaks. As a result, a number of companies have dedicated resources to the solution of the problem of leak detection.

The first level of protection against return line blood loss is the use of locking luers on all connections, as described in International Standard ISO 594-2 which help to minimize the possibility of spontaneous disconnection during treatment. Care in the connection and taping of lines to the patient's bodies is also a known strategy for minimizing this risk.

A higher level of protection is the provision of venous pressure monitoring, which detects a precipitous decrease in the venous line pressure. This technique is outlined in International Standard IEC 60601-2-16. This approach, although providing some additional protection, is not very robust, because most of the pressure loss in the venous line is in the needle used to access the patient. There is very little pressure change in the venous return line that can be detected in the event of a disconnection, so long as the needle remains attached to the return line. Thus, the pressure signal is very weak. The signal is no stronger for small leaks in the return line, where the pressure changes are too small to be detected with any reliability. One way to compensate for the low pressure signal is to make the system more sensitive, as described in U.S. Pat. No. 6,221,040, but this strategy can cause many false positives. It is inevitable that the sensitivity of the system will have to be traded against the burden of monitoring false alarms. Inevitably this leads to compromises in safety. In addition, pressure sensing methods cannot be used at all for detecting small leaks.

Yet another approach, described for example in PCT application US98/19266, is to place fluid detectors near the patient's access and/or on the floor under the patient. The system responds only after blood has leaked and collected in the vicinity of a fluid detector. A misplaced detector can defeat such a system and the path of a leak cannot be reliably predicted. For instance, a rivulet of blood may adhere to the patient's body and transfer blood to points remote from the detector. Even efforts to avoid this situation can be defeated by movement of the patient, deliberate or inadvertent (e.g., the unconscious movement of a sleeping patient).

Still another device for detecting leaks is described in U.S. Pat. No. 6,044,691. According to the description, the circuit is checked for leaks prior to the treatment operation. For example, a heated fluid may be run through the circuit and its leakage detected by means of a thermistor. The weakness of this approach is immediately apparent: there is no assurance that the system's integrity will persist, throughout the treatment cycle, as confirmed by the pre-treatment test. Thus, this method also fails to address the entire risk.

Yet another device for checking for leaks in return lines is described in U.S. Pat. No. 6,090,048. In the disclosed system, a pressure signal is sensed at the access and used to infer its integrity. The pressure wave may be the patient's pulse or it may be artificially generated by the pump. This approach cannot detect small leaks and is not very sensitive unless powerful pressure waves are used, in which case the effect can produce considerable discomfort in the patient.

Currently, with lower staffing levels comes the increased risk of unattended leaks. Thus, there has been, and continues to be, a need in the prior art for a foolproof approach to detection of a return line leak or disconnection.

In single-access systems, loss of blood through the patient access and blood circuit can be indirectly detected by detecting the infiltration of air during the draw cycle. Air is typically detected using an ultrasonic air detector on the tubing line, which detects air bubbles in the blood. The detection of air bubbles triggers the system to halt the pump and clamp the line to prevent air bubbles from being injected into the patient. Examples of such systems are described in U.S. Pat. Nos. 3,985,134, 4,614,590, and 5,120,303.

One type of double access is provided by a two-lumen needle or catheter, such as described in U.S. Pat. No. 4,202,332 and U.S. Pat. No. 4,144,884, which are hereby incorporated by reference as if fully set forth in their entireties herein. These use a single access point, for example a fistula. Blood is returned and drawn through a coaxial pair of channels at the end of the catheter or needle.

### SUMMARY OF THE INVENTION

Briefly, the inventions ensures against loss of integrity of a negative pressure venous connection to a patient due to improper mating of an access needle or catheter or loss of insertion of the same. The venous line may be connected to the access device permanently, so that only a pull-out of the access device can result in loss of blood through the venous line. The access device is constructed to ensure that such a pull-out will cause air to be sucked into the arterial line. Alternatively, a double connector mating arterial and venous lines to the access device ensures that an arterial seal will be lost if a venous seal is broken by improper fastening of the connector.

According to an embodiment, the invention provides a fluid circuit for use in extracorporeal blood treatment machines having air sensors for detecting air in arterial lines of fluid circuits connected thereto. The circuit includes a dual channel access device having venous and arterial channels. The access device may be a needle or a catheter or any suitable similar device. An arterial line is connected to the arterial channel and a venous line is connected to the venous channel. The venous line and the venous channel are configured such that the venous channel cannot become open to the air without exposing the arterial channel by either extraction of the access device or disconnection thereof. This assures that if there is a failure of integrity of the access, air will be drawn by the arterial line during operation to be detected by an air sensor a blood treatment machine. The venous line may be permanently connected to the access device arterial channel. Alternatively, a double connector may be used to mate the venous channel with the venous line and the arterial channel with the arterial line where the double connector assures the arterial line integrity will be lost if the venous line integrity is lost. For example, if there is less than full connection of the venous line with the venous channel, then the arterial line will not be connected and air will be drawn.

According to another embodiment, the invention provides a fluid circuit for use in extracorporeal blood treatment machines with air sensors for detecting air in arterial lines of fluid circuits connected thereto includes a dual channel access device having venous and arterial channels. The access device may be a needle or a catheter. An arterial line is connected to the access device arterial channel and a venous line connected to the venous channel. The venous line and the venous channel are configured to define a continuous permanent path free of intervening connectors up to a point in a flow path of the fluid circuit that is configured to be placed in operating association with an air sensor and under negative pressure.

According to yet another embodiment, the invention provides a fluid circuit for use in extracorporeal blood treatment machines with air sensors for detecting air in arterial lines of fluid circuits connected thereto. The fluid circuit has a dual channel access device having venous and arterial channels. An arterial line connects the access device arterial channel and a venous line connects to the venous channel using a double connector mating the venous channel with the venous line and the arterial channel with the arterial line. The connector is configured such that a loss of integrity of a connection between the arterial channel and the arterial line is ensured if there is a loss of integrity, by less than full connection of the connector, of a connection of the venous line with the venous channel.

### BRIEF DESCRIPTION OF THE DRAWINGS

FIG. 1 illustrates a blood processing machine.

FIG. 2 illustrates a fluid circuit with a dual access device attached thereto.

FIG. 3 illustrates features of the device of FIG. 2.

FIGS. 4 and 5 illustrate a double access device with a double connector that prevents a venous line disconnect with assuring an arterial line disconnect.

### DETAILED DESCRIPTION

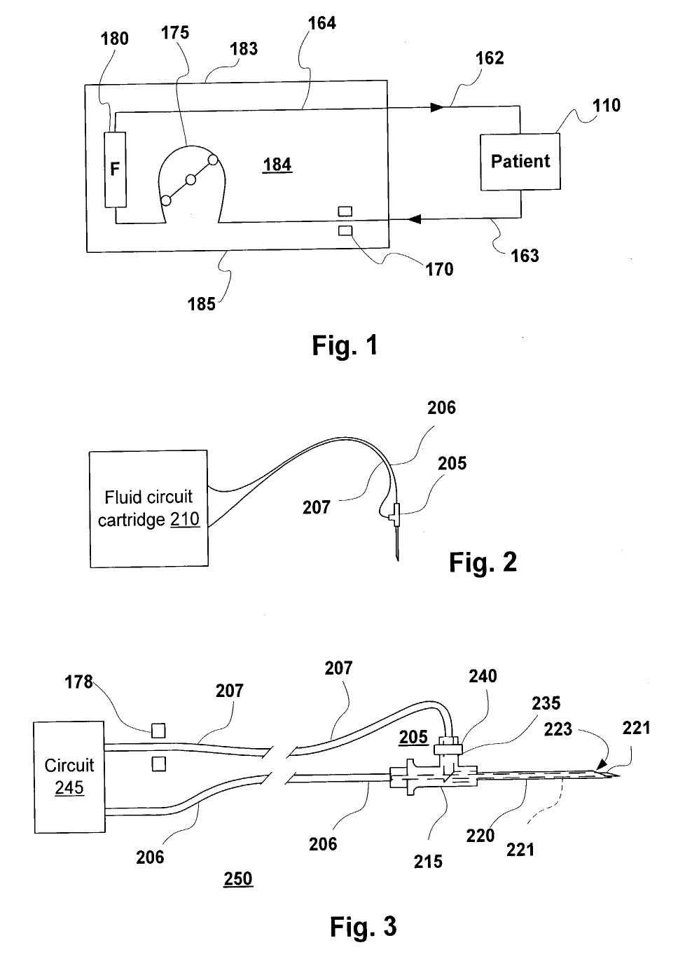

Referring now to FIG. 1, a blood processing machine 183 contains an air sensor 170, a filter 180, and a pump 175 among other components. The blood processing machine may be any of a variety of extracorporeal processing devices including hemofiltration, hemodialysis, and other enumerated in the Background Section. The machine 183 draws blood from a patient 110 through an arterial line 163 which passes through the air sensor 170. The blood passes through the filter 180 and is returned to the patient 110 through a venous line 162. If a disconnection of the arterial line occurs, the negative pressure caused by the pump will cause air to be drawn into the arterial line 163 which will immediately be detected by the air sensor 170. This is a known leak detection mechanism. However, if the venous line 162 becomes disconnected, considering the positive pressure in the venous line 162 and the direction of flow of blood therethrough, it is evident that detection of air infiltration will not protect against loss of blood.

Referring also to FIGS. 2 and 3, a double-access to a patient (not shown in the present figure) is provided by a needle or catheter 205 (the drawing is illustrative of a needle, but the same concepts to be discussed hereon apply to a catheter as will be evident). An arterial line 207, protected against disconnection, as discussed above, by an air sensor, draws blood through an outer cannula 220 through an annular space 223 between the outer cannula 220 and a concentric inner cannula 221. The inner cannula 221 injects blood into the patient access, for example a fistula.

The inner cannula 221 is permanently connected to the venous line 206 by way of a needle body 215 with internal branches which may be as described in either of the applications incorporated by reference above. The flow in the annular space 223 is directed by way of the internal branches to the arterial line 230 through a connector 240. The connector 240 may or may not be present, but the connection between the inner cannula 221 and the venous line is permanent. FIG. 2 illustrates a fluid circuit portion 210 in the form of a cartridge. Examples are illustrated in U.S. patent application Ser. No. 09/513,773, which is hereby incorporated by reference as if fully set forth in its entirety herein.

In the field of extracorporeal blood treatment, it is the general practice to provide connectors for connecting and disconnecting the venous line from the access device. Thus, the embodiment of FIG. 2 shows a consumable component that may be provided for treatment in which a permanent connection exists between the venous line 206 and a venous channel of the access device 205 of a consumable fluid circuit or portion thereof. FIG. 2 illustrates a cartridge, but the consumable device of FIG. 2 may be packaged in a form other than a cartridge, as is known.

In the embodiment of FIG. 3, the venous line 231 is permanently connected to the body 215 by, for example, adhesive bonding, thermal welding, mechanical lock or some other means. In a preferred embodiment, the permanent connection runs at least to a point of the air sensor 178 in the blood treatment machine 210. The permanent connection between the needle or catheter 205 may be ensured by providing the permanent arrangement as part of a fluid circuit with other portions 245 attached to the venous line 206 and ensuring the permanent connection is continuous up to and including the pump portion of the circuit 175 and a first air sensor 170. This ensures that if any connections are improperly made, they can only happen in such a way as to cause air to be drawn in. To summarize, a fluid circuit 250 defines a continuous and permanent connection from an entry point 223 up to an air sensor 178. The circuit may include various portions 245 which may include a filter 180 and other components for a blood treatment. Thus, the filter 180 (embedded in portion 245), in a preferred configuration, is permanently attached and supplied with the fluid circuit 250.

Note that preferably the needle or catheter 205 is of such design that if the needle pulls out, there is no practical possibility that the venous line 206 could allow blood loss without air being sucked into the arterial line. The permanent connection is a part of this, but if the venous cannula extends further than the arterial cannula, this can be assured.

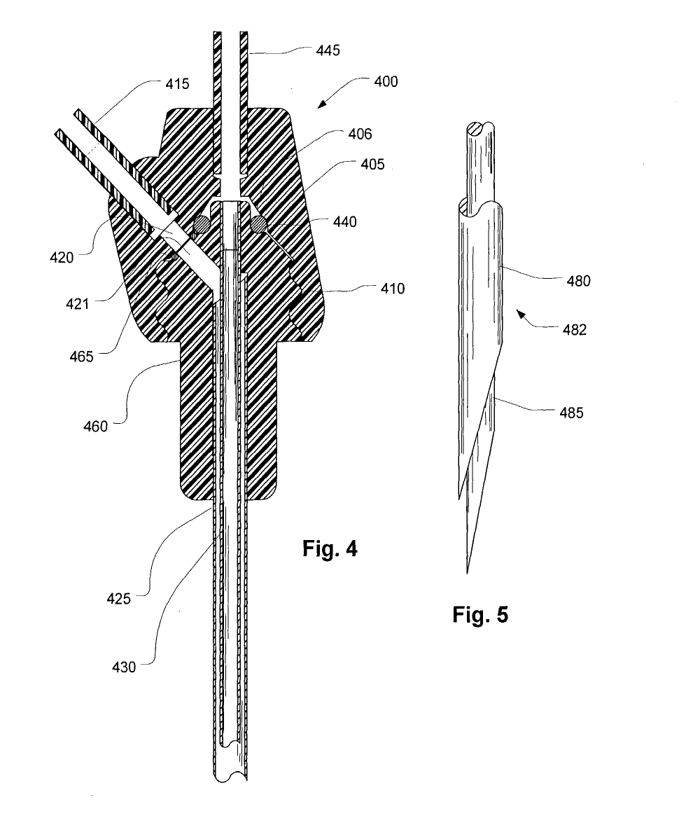

Referring to FIGS. 4 and 5, a removable connector 400 for a double access needle or catheter ensures that it is virtually impossible for the venous channel to become disconnected without the arterial channel becoming disconnected, thereby ensuring that air will be detected and conventional safeguards will prevent blood loss. Female 405 and male 460 parts mate arterial 415 and venous 445 lines with arterial 425 and venous cannulae, respectively. A high pitch thread 410 forces the male part 460 into the female part 405 mating a venous O-ring seal 440 and an arterial O-ring seal 465. The venous O-ring seal includes a sloped recess 406 that compresses the O-ring 440 progressively as two halves of an arterial channel 420 and 421 are brought into alignment and sealed by the arterial O-ring seal 465. It should be evident that the configuration can provide that the venous O-ring seal 440 cannot fail to be made by improper connection without the arterial O-ring seal 465 not being made. The shape of the threads 410 are preferably made to ensure that air can pass through and into the arterial line at any time if the arterial O-ring seal 465 is not made. Preferably, the venous cannula (or catheter portion) 485 should extend further than the arterial cannula (or catheter portion) 480 as illustrated in FIG. 5. This is so that the arterial passage will be open to air if the venous passage is broken by the needle 482 coming of a patient access.

What is claimed is:

1. A fluid circuit for use in extracorporeal blood treatment machines having air sensors for detecting air in arterial lines of fluid circuits connected thereto, comprising:
a dual channel access device having venous and arterial channels;
said access device including one of a needle and a catheter;
an arterial line connected to said access device arterial channel and a venous line connected to said venous channel, said venous line and said venous channel being configured such that said venous channel cannot become open to the air without exposing the arterial channel by either extraction of the access device or disconnection thereof,
whereby a failure of integrity of said access will permit air to be drawn by said arterial line during operation to be detected by an air sensor a blood treatment machine.

2. A fluid circuit as in claim 1, wherein said venous line is permanently connected to said access device arterial channel.

3. A fluid circuit as in claim 1, with a double connector mating said venous channel with said venous line and said arterial channel with said arterial line, said connector being configured such that a loss of integrity of a connection between said arterial channel and said arterial line is ensured if there is a loss of integrity, by less than full connection of said connector, of a connection of said venous line with said venous channel.

4. A fluid circuit as in claim 3, wherein said access device includes a dual lumen catheter.

5. A fluid circuit as in claim 3, wherein said access device includes a dual lumen needle.

6. A fluid circuit as in claim 1, wherein said access device includes a dual lumen catheter.

7. A fluid circuit as in claim 6, wherein said venous line is permanently connected to said access device arterial channel.

8. A fluid circuit as in claim 1, wherein said access device includes a dual lumen needle.

9. A fluid circuit as in claim 8, wherein said venous line is permanently connected to said access device arterial channel.

10. A fluid circuit for use in extracorporeal blood treatment machines having air sensors for detecting air in arterial lines of fluid circuits connected thereto, comprising:
a dual channel access device having venous and arterial channels;
said access device including one of a needle and a catheter;
an arterial line connected to said access device arterial channel and a venous line connected to said venous channel, said venous line and said venous channel being configured to define a continuous permanent path free of intervening connectors up to a point in a flow path of said fluid circuit that is configured to be placed in operating association with an air sensor and under negative pressure.

11. A fluid circuit for use in extracorporeal blood treatment machines having air sensors for detecting air in arterial lines of fluid circuits connected thereto, comprising:
a dual channel access device having venous and arterial channels;
said access device including one of a needle and a catheter;
an arterial line connected to said access device arterial channel and a venous line connected to said venous channel, said venous line and said venous channel being configured such that said venous channel cannot become open to the air without exposing the arterial channel by either extraction of the access device or disconnection thereof,
a double connector mating said venous channel with said venous line and said arterial channel with said arterial line, said connector being configured such that a loss of integrity of a connection between said arterial channel and said arterial line is ensured if there is a loss of integrity, by less than full connection of said connector, of a connection of said venous line with said venous channel.
